SEQUENCE LISTING (3 sequences)

SEQ ID NO: 1 - protein, 215 aa, Homo sapiens
MCTEGAFPHRSACSLPLTHVHTHIHVCVPVLWGSVPRGMKLQCVSLWLLGTILILCSVDNHGLRRCLISTDMHHIEESFQEIKRAIQAKDTFPNVTILSTLETLQIIKPLDVCCVTKNLLAFYVDRVFKDHQEPNPKILRKISSIANSFLYMQKTLRQCQEQRQCHCRQEATNATRVIHDNYDQLEVHAAAIKSLGELDVFLAWINKNHEVMFSA

SEQ ID NO: 2 - protein, 177 aa, Homo sapiens
MKLQCVSLWLLGTILILCSVDNHGLRRCLISTDMHHIEESFQEIKRAIQAKDTFPNVTILSTLETLQIIKPLDVCCVTKNLLAFYVDRVFKDHQEPNPKILRKISSIANSFLYMQKTLRQCQEQRQCHCRQEATNATRVIHDNYDQLEVHAAAIKSLGELDVFLAWINKNHEVMFSA

SEQ ID NO: 3 - protein, 159 aa, Homo sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

(19) United States
(12) Patent Application Publication
(10) Pub. No.: US 2015/0104465 A1
(43) Pub. Date: Apr. 16, 2015

(54) IL-19 AS A BIOMARKER OF TSLP TREATMENT

(71) Applicant: Merck Sharp & Dohme Corp., Rahway, NJ (US)

(72) Inventor: Pia Bjorck, Menlo Park, CA (US)

(21) Appl. No.: 14/354,070

(22) PCT Filed: Oct. 24, 2012
(86) PCT No.: PCT/US12/61614

§ 371 Date: Dec. 23, 2014

Related U.S. Application Data
(60) Provisional application No. 61/552,617, filed on Oct. 28, 2011.

(51) Int. Cl. G01N 33/68; C07K 16/24

(52) U.S. Cl. CPC G01N 33/6869; C07K 16/24; G01N 2800/52; G01N 2800/122; G01N 2800/202; C07K 2317/76
USPC 424/158.1

(57) ABSTRACT

The present invention relates to the use of IL-19 as a biomarker of treatment with a TSLP antagonist.

### FIELD OF THE INVENTION

The present invention relates to the use of IL-19 as a biomarker of treatment with a TSLP antagonist.

### BACKGROUND OF THE INVENTION

TSLP is an immune cytokine that induces dendritic cell-mediated CD4⁺ T cell responses with a proallogenic phenotype. Dendritic cells activated by TSLP play crucial role in the induction and maintenance of allergic inflammatory Th2 by production of proallergenic cytokines, chemokines and costimulatory molecules that direct naïve T cells to become Th2 cells, producing IL-4, IL-5 and IL-13. Over-expression of TSLP in Atopic Dermatitis (AtD), Netherton Syndrome and asthma indicates a crucial role of this cytokine in the pathogenesis of these allergic inflammatory diseases. The use of TSLP antagonists for the treatment of allergic disease is under clinical investigation. The need exists for methods for monitoring the efficacy of treatment with TSLP antagonists. Such methods would preferably allow objective determination of a subject's disease state and/or response to treatment with a TSLP antagonist.

A “biomarker” is an objectively measured indicator that reflects the presence, progression, or successful treatment of a particular condition. Biomarkers have long been used in drug development, and the discovery and validation of new efficacy biomarkers is expected to improve the predictive disease models, reduce the time and cost associated with drug development, and increase the success rate of translating experimental drugs into clinical therapeutics. In addition, biomarkers are valuable in early detection of disease development, changes in disease status, and effectiveness of behavioral modifications and therapeutics in disease control.

IL-19 belongs to the IL-10 family of cytokines, which also includes IL20, MDA7, and IL22. By searching EST databases using IL10 as probe, followed by screening an Epstein-Barr virus-transformed B-cell cDNA library, Gallagher et al. obtained a cDNA encoding IL19. Gallagher et al., Genes Immunity 1: 442-450, (2000). See also U.S. Pat. No. 7,056,681.

### SUMMARY OF THE INVENTION

The present invention meets these needs in the art by providing the use of IL-19 as a biomarker whose level reflects response to treatment with a TSLP antagonist.

In one embodiment, the invention relates to methods of monitoring TSLP blockade (i.e. target engagement) by a therapeutic agent. Such methods may find use in clinical trials and also in the clinic after regulatory approval. The therapeutic agent may be an antagonist of TSLP or an antagonist of the TSLP receptor (for example, an anti-TSLP or anti-TSLPR antagonistic antibody). In a clinical trial, monitoring TSLP blockade is useful in determining whether any failure to achieve therapeutic benefit, if observed, is caused by failure to engage the TSLP pathway, as opposed to failure of the intervention in the target pathway to effect a therapeutic benefit. In the clinic, validation of TSLP blockade is useful in determining whether a therapeutic agent is active, for example whether a drug retains its desired biological activity, for example in subjects that do not exhibit apparent benefit or symptom relief.

In one embodiment, the invention provides a method for monitoring TSLP blockade in a mammalian subject treated with a TSLP antagonist comprising: measuring expression of IL-19 in a sample from said subject; wherein decreased expression of IL-19 compared to a control is indicative of TSLP blockade in the subject and suitability of said subject for treatment with a TSLP antagonist; or wherein unchanged or higher expression of IL-19 compared to a control is indicative of a lack of TSLP blockade in response to a TSLP antagonist in the subject and a lack of suitability of said subject for treatment with a TSLP antagonist.

In one embodiment, the invention provides a method for monitoring TSLP blockade in a mammalian subject treated with a TSLP antagonist comprising: a) measuring expression of IL-19 in a sample from said subject; and b) comparing the expression of IL-19 from step a) to a control, wherein decreased expression of IL-19 compared to a control is indicative of TSLP blockade in the subject and suitability of said subject for treatment with a TSLP antagonist; or wherein unchanged or higher expression of IL-19 compared to a control is indicative of a lack of TSLP blockade in response to a TSLP antagonist in the subject and a lack of suitability of said subject for treatment with a TSLP antagonist.

In another embodiment, the invention provides a method for monitoring TSLP blockade in a mammalian subject treated with a TSLP antagonist comprising: a) obtaining a baseline biological sample from the subject prior to administering a dose of a TSLP antagonist; b) measuring the expression of at IL-19 in the baseline biological sample, c) obtaining from the subject at least one subsequent biological sample after the subject has been administered a TSLP antagonist; d) measuring the expression of IL-19 in the subsequent sample; e) comparing the expression of IL-19 in the subsequent biological sample with the level of IL-19 in the baseline biological sample, wherein a decrease in expression of IL-19 in the subsequent biological sample indicates TSLP blockade.

In another embodiment, the invention provides a method for monitoring TSLP blockade in a mammalian subject treated with a TSLP antagonist comprising: a) obtaining a baseline biological sample from the subject prior to administering a dose of a TSLP antagonist; b) measuring the expression of at IL-19 in the baseline biological sample, c) administering the a TSLP antagonist to the subject; d) obtaining from the subject at least one subsequent biological sample; e) measuring the expression of IL-19 in the subsequent sample; f) comparing the expression of IL-19 in the subsequent biological sample with the level of IL-19 in the baseline biological sample, wherein a decrease in expression of IL-19 in the subsequent biological sample indicates TSLP blockade.

In another aspect of the invention, IL-19 is used to track disease progression in subjects undergoing treatment with a TSLP antagonist. The disease may be an allergic disease, such as asthma or atopic dermatitis. The dose, dosing frequency (interval) or other therapeutic parameter may be modified based at least partly on the expression of IL-19 to ensure that the patient achieves a satisfactory outcome. Such methods are objective, based the results of lab tests rather than the subjective assessment of symptoms by the patient. Patients may be, for example, subjects in a clinical trial or patients receiving an approved treatment.

The invention comprises a method for detecting the expression level of IL-19 in a sample from a subject treated with a TSLP antagonist, comprising: measuring expression of IL-19 in a sample the said subject; wherein decreased expression of IL-19 compared to a control is indicative of the presence of a beneficial response in the patient; or wherein unchanged or increase expression of IL-19 compared to a control is indicative of the absence of a beneficial response in the patient.

The invention also comprises a method for monitoring TSLP blockade in a subject treated with a TSLP antagonist comprising: measuring expression of IL-19 in a sample from the subject; wherein decreased expression of IL-19 compared to a control is indicative of TSLP blockade in the subject; or wherein unchanged or higher expression of IL-19 compared to a control is indicative of a lack of TSLP blockade in response to a TSLP antagonist in the subject

In one embodiment the invention provides a method for detecting the presence or absence of a beneficial response in a subject after administration of a TSLP antagonist, comprising: a) measuring expression of IL-19 in a sample from said subject; and b) comparing the expression of IL-19 from step a) to a control; wherein decreased expression of IL-19 compared to a control is indicative of the presence of a beneficial response in a patient or wherein unchanged or increase expression of IL-19 compared to a control is indicative of the absence of a beneficial response in a patient.

In another embodiment, the invention provides a method of treating an allergic disease in a mammalian subject in need thereof comprising measuring the expression IL-19 in a subject suffering from allergic disease at a first timepoint, administering a TSLP antagonist, re-measuring the expression of IL-19 at a second timepoint, comparing the results of the first and second timepoints, and modifying the treatment regimen based on the comparison. In one embodiment, the first timepoint is prior to administration of a TSLP antagonist, and the second timepoint is after said administration of the TSLP antagonist. In one embodiment, the first timepoint is prior to the administration of the TSLP antagonist to the subject for the first time. In one embodiment, the dose (defined as the quantity of TSLP antagonist agent administered at any one administration) is increased or decreased in response to the comparison. In another embodiment, the dosing interval (defined as the time between successive administrations) is increased or decreased in response to the comparison, including total discontinuation of treatment. Dosing interval is inversely related to dosing frequency. In one embodiment, the allergic disease is atopic dermatitis. In another embodiment, the allergic disease is asthma.

The invention also comprises a method of treating allergic disease in a mammalian subject in need thereof, the method comprising the steps of: a) administering an effective amount of a TSLP antagonist to the subject; b) comparing the expression of IL-19 to a control; and c) and, if IL-19 levels in the sample are higher than a control, administering a therapeutically effective amount of a TSLP antagonist to the subject. In one embodiment, the allergic disease is atopic dermatitis. In another embodiment, the allergic disease is asthma.

The invention also comprises a method of treating an allergic disease, the method comprising the steps of: administering an effective amount of a TSLP antagonist to a subject in need thereof; wherein the subject, prior to the administration of the TSLP antagonist, has been tested for expression of IL-19; wherein expression level of IL-19 relative to a control guides the determination of whether treatment with a TSLP antagonist should be continued, discontinued or modified. In one embodiment, the allergic disease is atopic dermatitis. In another embodiment, the allergic disease is asthma.

In one embodiment, the invention provides a method of treating an allergic disease in a subject in need thereof, the method comprising the steps of: a) administering an effective amount of a TSLP antagonist; b) measuring expression of IL-19 in a sample from said subject; and c) comparing the expression of IL-19 from step b) to a control; and, if IL-19 levels in the sample are higher than a control, administering a therapeutically effective amount of a TSLP antagonist to the subject. In one embodiment, the allergic disease is atopic dermatitis. In another embodiment, the allergic disease is asthma.

In one embodiment, the invention provides a method of treating an allergic disease in a subject in need thereof, the method comprising the steps of: measuring expression of IL-19 in a sample from said subject and, if IL-19 levels in the sample are higher than a control, administering a therapeutically effective amount of a TSLP antagonist to the subject. In one embodiment, the allergic disease is atopic dermatitis. In another embodiment, the allergic disease is asthma.

The invention also comprises a method for treating a subject with a TSLP antagonist comprising: a) obtaining a first biological sample from the subject prior to administering a dose of a TSLP antagonist; b) measuring the expression of IL-19 in the baseline biological sample, c) administering the TSLP antagonist to the subject; d) obtaining from the subject a second biological sample; e) measuring the expression of IL-19 in the second sample; f) comparing the expression of IL-19 in the second biological sample with the expression of IL-19 in the first biological sample, wherein, if IL-19 levels are reduced in the second biological sample as compared to the first biological sample, administering a therapeutically effective amount of a TSLP antagonist to the subject.

In another aspect, the invention relates to methods of selecting subjects, e.g. allergic disease patients, for treatment with a TSLP antagonist. In one embodiment, the invention comprises a method for selecting a subject for treatment with a TSLP antagonist comprising: a) measuring expression of IL-19 in a sample from said subject and, if IL-19 levels in the sample are higher than the levels of a control, administering a therapeutically effective amount of a TSLP antagonist to the subject. In one embodiment, the subject has an allergic disease. In one embodiment, allergic disease is atopic dermatitis. In another embodiment, the allergic disease is asthma.

In another embodiment, the invention comprises a method for selecting a subject for treatment with a TSLP antagonist comprising: a) measuring expression of IL-19 in a sample from said subject; and b) determining whether subject should be treated with a

TSLP antagonist. In one embodiment, the subject has asthma. In another embodiment, the subject has atopic dermatitis. An elevated level of IL-19 (compared to a control) in the sample may suggest that the subject would be a good candidate for treatment with a TSLP antagonist, such as an anti-TSLP or anti-TSLPR antibody.

The invention also comprises a method for monitoring progress of treatment with a TSLP antagonist comprising: a) measuring expression of IL-19 in a sample from said subject, and, if IL-19 levels in the sample are higher than the levels of a control, then continuing to administer a therapeutically effective amount of a TSLP antagonist to the subject. In one embodiment, the subject has an allergic disease. In one embodiment, allergic disease is atopic dermatitis. In another embodiment, the allergic disease is asthma.

The invention also provides the use of IL-19 as a biomarker for allergic disease. In one aspect, the invention provides the use of IL-19 as a biomarker to assess the progression or disease state of allergic disease.

The invention also provides the use of IL-19 as a biomarker for atopic dermatitis. In one aspect, the invention provides the use of IL-19 as a biomarker to assess the progression or disease state of atopic dermatitis.

The invention also provides the use of IL-19 as a biomarker for asthma. In one aspect, the invention provides the use of IL-19 as a biomarker to assess the progression or disease state of asthma.

In various embodiments, the TSLP antagonist is an antibody or antigen binding fragment thereof that binds to TSLP or the receptor for TSLP (or any subunit of the TSLP receptor). In various embodiments the antibody is a polyclonal, monoclonal, chimeric, humanized or fully human antibody. In other embodiments the TSLP antagonist is a nucleic acid molecule, such as an antisense nucleic acid or an siRNA, targeting TSLP or its receptor, or any subunit of either. In still further embodiments the TSLP antagonist is a small molecule compound.

In one embodiment, the expression of IL-19 is detected in blood (including plasma or serum).

In another embodiment, the expression of IL-19 is detected in a tissue sample.

In another embodiment, the expression of IL-19 is detected in a skin biopsy.

In another embodiment, the expression of IL-19 is detected in sputum.

In some embodiments the method involves detection of biomarker protein levels by immunological detection means, such as ELISA, immunohistochemistry (IHC), fluorescence activated cell sorting (FACS®), Western blot or other immunologically based protein detection method. In other embodiments, the biomarker protein is determined by non-immunological detection means, such as by mass spectroscopic or chromatographic means. In yet other embodiments biomarkers are measured at the gene expression level by gene expression detection means, such as by detecting the level of mRNA, for example using a nucleic acid hybridization-based technique (e.g. an array or chip) or an amplification-based technique (e.g. polymerase chain reaction, TagMan® real time quantitative PCR analysis).

In other embodiments, the level of IL-19 is judged to be higher or lower than the level in a pre-determined reference sample or control (e.g. from subject(s) not having an inflammatory disease (such as atopic dermatitis or asthma) or from the subject prior to receiving treatment with a TSLP antagonist) if it differs by a specified multiple, such as 1.1-, 1.2-, 1.3-, 1.4-, 1.5-, 2-, 3-, 4-, 5-, 7-, 10-, 15-, 20-, 50-, 100-fold or more.

### BRIEF DESCRIPTION OF THE DRAWINGS

FIG. 1 shows that calcipotriol-induced skin inflammation is reduced in IL-19KO mice treated with an anti-TSLP antibody.

FIG. 2 shows that IL-19 was detected at high levels in serum of cacipotriol treated mice and that an anti-TSLP antibody suppresses the secretion of IL-19 but not IL-20.

FIG. 3 shows that the serum of TSLPKO mice treated with calipotriol contain little or no IL-19.

FIG. 4 shows that the inhibition of IL-19 in serum of calcipotriol treated mouse skin is dependent on the dose of the anti-TSLP antibody administered.

FIG. 5 shows the kinetics of IL-19 secretion in the serum of calcipotriol treated mice, and shows the time- and dose dependent inhibition of IL-19 by an anti-TSLP antibody.

FIG. 6 shows real-time qPCR (Taqman) analysis of skin from calcipotriol treated mice showing that the mRNA of both IL-19 and its receptor (IL20R1/IL20R2) is induced on day 4 of calcipotriol treatment.

FIG. 7 shows the expression of IL-19 in human skin histoculture supernatants induced to secrete TSLP.

FIG. 7 shows the effect of neutralizing anti-TSLP antibodies on the expression of IL-19 in human skin histoculture supernatants.

### DETAILED DESCRIPTION

As used herein, including the appended claims, the singular forms of words such as “a,” “an,” and “the,” include their corresponding plural references unless the context clearly dictates otherwise.

All references cited herein are incorporated by reference to the same extent as if each individual publication, database entry (e.g. GenBank sequences or GeneID entries), patent application, or patent, was specifically and individually indicated to be incorporated by reference. GenBank accession numbers for nucleic acid and protein sequences referenced herein refer to the contents of the database as of the filing date of this application. Although such database entries may be subsequently modified, GenBank maintains a public record of all prior versions of the sequences as a function of date, making such database entries an unambiguious reference to a specific sequence.

This statement of incorporation by reference is intended by Applicants, pursuant to 37 C.F.R. §1.57(b)(1), to relate to each and every individual publication, database entry (e.g. GenBank sequences or GeneID entries), patent application, or patent, each of which is clearly identified in compliance with 37 C.F.R. §1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

### I. Definitions

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding composition, or by another cell. “Treatment,” as it applies to a human, veterinary, or research subject, may refer to therapeutic treatment, prophylactic or preventative measures, to research and diagnostic applications. For a relapsing/remitting-type disease like IBD, a treatment that prevents, delays or reduces severity of a relapse can be said to either “treat” the overall disease or to prophylactically “prevent” the relapse, and as such the distinction between treatment and prophylaxis is difficult. As use herein, “treatment” refers to reduction of signs or symptoms, or reduction of duration or severity, of an IBD episode active during the start of therapy, whereas “prevention” refers to the prevention, delay or reduction of severity of an IBD episode beginning after the start of therapy, although any given therapeutic regimen may be constitute both treatment and prevention as used herein. “Treatment” as it applies to a human, veterinary, or research subject, or cell, tissue, or organ, encompasses contact of an agent with animal subject, a cell, tissue, physiological compartment, or physiological fluid. “Treatment of a cell” also encompasses situations where the agent contacts TSLP or its receptor, e.g., in the fluid phase or colloidal phase.

As used herein, “subject” refers to a specific individual, usually a human, of interest. A “subject” may be a human subject who is diagnosed with, or suspected of having, a disease or disorder and/or is under treatment for a disease or disorder. The term “subject” and “patient” are used interchangeably in this application.

As used herein, “biological sample” may comprise any sample obtained from a subject, including but not limited to whole blood, plasma, serum, tissue biopsy (e.g., lung or skin), sputum, bronchoalveolar lavages (BAL) cells, nasal exudate, nasal scrape or urine.

As used herein, the “expression” or “level” of IL-19 relates to the amount of IL-19 polypeptide present in a sample or the amount of mRNA encoding IL-19 present in a sample.

As used herein “monitoring” refers to measuring and/or recording changes in a varying parameter.

As used herein, the term “antibody” may refer to any form of antibody that exhibits the desired biological activity. Thus, it is used in the broadest sense and specifically covers monoclonal antibodies (including full length monoclonal antibodies), polyclonal antibodies, multispecific antibodies (e.g., bispecific antibodies), chimeric antibodies, humanized antibodies, fully human antibodies, antibody fragments, etc. so long as they exhibit the desired biological activity.

Antibody fragments include Fab, Fab′, F(ab′)₂, and Fv fragments; diabodies; linear antibodies; single-chain antibody molecules, e.g., sc-Fv; domain antibodies; and multispecific antibodies formed from antibody fragments. Typically, an antibody fragment or derivative retains at least 10% of its affinity for its target, e.g. no more than a 10-fold change in the dissociation equilibrium binding constant (IQ). Preferably, an antibody fragment or derivative retains at least 25%, 50%, 60%, 70%, 80%, 90%, 95%, 99% or 100% (or more) of its binding affinity, although any binding fragment with sufficient affinity to exert the desired biological effect will be useful. It is also intended that, when specified, an antibody fragment can include sequence variants with conservative amino acid substitutions that do not substantially alter its biologic activity.

A “TSLP antagonist” is a molecule that inhibits the activity of TSLP in any way. In some embodiments, a TSLP antagonist is an antibody or antigen binding fragment that inhibits TSLP signaling via the TSLP receptor, for example by binding to TSLP or its receptor. In other embodiments a TSLP antagonist is a small molecule or a polynucleotide, such as an antisense nucleic acid or siRNA. In another embodiment, the TSLP antagonist is a soluble TSLP receptor or a TSLP-IgG fusion protein.

Monoclonal antibodies specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. U.S. Pat. No. 4,816,567; Morrison et al. (1984) Proc. Natl. Acad. Sci. USA 81: 6851-6855.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

A “bivalent antibody” comprises two antigen binding sites. In some instances, the two binding sites have the same antigen specificities. However, bivalent antibodies may be bispecific.

As used herein, the term “single-chain Fv” or “scFv” antibody refers to antibody fragments comprising the V_{H} and V_{L} domains of antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv, see Pluckthun (1994) THE PHARMACOLOGY OF MONOCLONAL ANTIBODIES, vol. 113, Rosenburg and Moore eds. Springer-Verlag, N.Y. pp. 269-315.

The monoclonal antibodies herein also include camelized single domain antibodies. See, e.g., Muyldermans et al. (2001) Trends Biochem. Sci. 26:230; Reichmann et al. (1999). J. Immunol. Methods 231:25; WO 94/04678; WO 94/25591; U.S. Pat. No. 6,005,079). In one embodiment, the present invention provides single domain antibodies comprising two V_{H} domains with modifications such that single domain antibodies are formed.

As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

As used herein, the term “humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Pc), typically that of a human immunoglobulin. The prefix “hum”, “hu” or “h” is added to antibody clone designations when necessary to distinguish humanized antibodies from parental rodent antibodies (although these same designations, depending on the context, may also indicate the human form of a particular protein). The humanized forms of rodent antibodies will generally comprise the same CDR sequences of the parental rodent antibodies, although certain amino acid substitutions may be included to increase affinity, increase stability of the humanized antibody, or for other reasons.

Antibodies also include antibodies with modified (or blocked) Fc regions to provide altered effector functions. See, e.g., U.S. Pat. No. 5,624,821; WO 2003/086310; WO 2013/063062 PCT/US2012/061614

WO 2005/120571; WO 2006/0057702; Presta (2006) Adv. Drug Delivery Rev. 58:640-656. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies. A longer half-life may result in less frequent dosing, with the concomitant increased convenience and decreased use of material. See Presta (2005) J. Allergy Clin. Immunol. 116:731 at 734-35.

Antibodies also include antibodies with intact Fc regions that provide full effector functions, e.g. antibodies of human isotype IgG1, which induce complement-dependent cytotoxicity (CDC) or antibody dependent cellular cytotoxicity (ADCC) in the a targeted cell.

The antibodies of the present invention also include antibodies conjugated to cytotoxic payloads, such as cytotoxic agents or radionuclides. Exemplary cytotoxic agents include ricin, vinca alkaloid, methotrexate, Psuedomonas exotoxin, saporin, diphtheria toxin, cisplatin, doxorubicin, abrin toxin, gelonin and pokeweed antiviral protein. Exemplary radionuclides for use in immunotherapy with the antibodies of the present invention include ¹²⁵I, ¹³¹I, ⁹⁰Y, ⁶⁷Cu, ²¹¹At, ¹⁷⁷Lu, ¹⁴³Pr and ²¹³Bi. See,. e.g., U.S. Patent Application Publication No. 2006/0014225.

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively. A fully human antibody may be generated in a human being, in a transgenic animal having human immunoglobulin germline sequences, by phage display or other molecular biological methods.

“Effective amount” encompasses an amount sufficient to ameliorate or prevent a symptom or sign of the medical condition. Such an effective amount need not necessarily completely ameliorate or prevent such symptom or sign. Effective amount also means an amount sufficient to allow or facilitate diagnosis. An effective amount for a particular patient or veterinary subject may vary depending on factors such as the condition being treated, the overall health of the patient, the method route and dose of administration and the severity of side effects. See, e.g., U.S. Pat. No. 5,888,530. An effective amount can be the maximal dose or dosing protocol that avoids significant side effects or toxic effects. An effective amount will typically result in an improvement of a diagnostic measure or parameter by at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%, where 100% is defined as the diagnostic parameter shown by a normal subject. See, e.g., Maynard et al. (1996) A Handbook of SOPs for Good Clinical Practice, Interpharm Press, Boca Raton, Fla.; Dent (2001) Good Laboratory and Good Clinical Practice, Urch Publ., London, UK.

“Allergic disease” refers to any disease caused by a hypersensitivity disorder of the immune system. It includes, without limitation, such as asthma, atopic dermatitis, Ichtyosis Prematurity Syndrome, allergic rhinitis, eosinophilic esophagitis, and Netherton Syndrome.

As used herein, “polymerase chain reaction” or “PCR” refers to a procedure or technique in which minute amounts of a specific piece of nucleic acid, RNA and/or DNA, are amplified as described in, e.g., U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al. (1987) Cold Spring Harbor Symp. Quant. Biol. 51:263; Erlich, ed., (1989) PCR TECHNOLOGY (Stockton Press, N.Y.). As used herein, PCR is considered to be one, but not the only, example of a nucleic acid polymerase reaction method for amplifying a nucleic acid test sample comprising the use of a known nucleic acid as a primer and a nucleic acid polymerase to amplify or generate a specific piece of nucleic acid.

“Specifically” or “selectively” binds, when referring to a ligand/receptor, antibody/antigen, or other binding pair, indicates a binding reaction that is determinative of the presence of the protein in a heterogeneous population of proteins and other biologics. Thus, under designated conditions, a specified ligand binds to a particular receptor and does not bind in a significant amount to other proteins present in the sample. As used herein, an antibody is said to bind specifically to a polypeptide comprising a given sequence if it binds to polypeptides comprising the polypeptide sequence but does not bind to proteins lacking the polypeptide sequence.

The antibody, or binding composition derived from the antigen-binding site of an antibody, of the contemplated method binds to its antigen with an affinity that is at least two fold greater, preferably at least ten times greater, more preferably at least 20-times greater, and most preferably at least 100-times greater than the affinity with unrelated antigens. In a preferred embodiment the antibody will have an affinity that is greater than about 10⁹ liters/mol, as determined, e.g., by Scatchard analysis. Munsen et al. (1980) Analyt. Biochem. 107:220-239.

### II. General

The present invention provides the use of IL-19 as a biomarker for TSLP treatment, or as a biomarker of allergic disease (for example, atopic dermatitis or asthma). IL-19 is a well known cytokine that belongs to the IL-10 cytokine subfamily. In humans, two different splice variants have been identified (GenBank Accession Nos. NM_{—}153758.2) and NM_{—}013371.3). As used herein, the term “IL-19” refers to either isoform of IL-19.

IL-19 can find use as a biomarker in several contexts. It may be used in diagnosing allergic disease (for example, atopic dermatitis or asthma) or in staging subjects for disease severity.

IL-19 can find use as a biomarker to select patient subpopulations likely to respond to treatment with a TSLP antagonists. The present invention demonstrates that IL-19 levels are reverted toward non-disease levels when animals are treated with a TSLP antagonist antibody. Accordingly, allergic subjects having elevated levels of IL-19 may be considered likely candidates for therapy with a TSLP antagonist to revert the levels of the biomarkers to non-disease levels. Conversely, allergic subjects without elevated levels of IL-19 may be poor candidates for treatment with a TSLP antagonist.

IL-19 may also find use as a biomarker in subjects undergoing treatment, for example with a TSLP antagonist, to confirm blockade of the TSLP pathway, to assess the efficacy of treatment (and modification of therapeutic regimen if necessary), and to monitor patient progress generally. If results demonstrate that a given therapeutic regimen effectively engages the target pathway in a patient, and yet fails to provide a therapeutic benefit, then it may be that TSLP signaling is of relatively little practical significance in the patient.

IL-19 may also find use as a biomarker in management of patients in the clinic, for example to inform modification of therapeutic regimen if necessary. A clinician may monitor the level of IL-19 to help decide whether dosing with a TSLP antagonist should be increased, decreased, or made more or less frequent, depending on the degree to which the patient is responding to existing therapy. Note that reduction of the frequency of administration may constitute a reduced “dose” in that the subject will receive less drug over a given period of time, when the timeframe is longer than a single dosing interval. Measurement of the levels of IL-19 has the advantage that it may be possible to determine which subjects are responding favorably to treatment with a TSLP antagonist at an earlier time (i.e. sooner after treatment) than would be possible using standard clinical disease measures, some of which rely at least in part on symptomatic relief. Early discrimination of responders from non-responders allows for earlier modification of dosing or discontinuation. Early modifications of the therapeutic regimen can reduce the time to successful treatment, or reduce the risk of unnecessary exposure to an ineffective drug (with concomitant reduction in expense and side-effects).

Assessment of the efficacy of a given therapeutic regiment is important for management of patient care, and essential for evaluation of potential therapeutic agents, as in clinical trials.

### III. TSLP Antagonists

Allergic diseases may be treated using antagonists of TSLP. Antagonists of TSLP include agents, such as antibodies or fragments thereof, which bind to TSLP or its receptor. The sequence of human TSLP is found, for example, at GenBank Accession No: CBX74361.

The TSLP receptor is composed of two subunits: TSLPR (CRLF2) and IL7Ralpha subunits. Reche et al., J. Immun. 167: 336-343 (2001).

In one embodiment, the TSLP antagonist is an anti-human TSLP antibody comprises the heavy and light chain variable domains of the humanized antibodies disclosed in commonly assigned International Pat. Appl. Pub. No. WO 2008/076321 or WO2011/056772.

In various embodiments the TSLP antagonists of the present invention comprise antigen binding fragments of antibodies, such as fragments of any of the TSLP antagonist antibodies referred to herein. Such fragments include, but are not limited to, Fab, Fab′, Fab′-SH, Fv, scFv, F(ab′)₂, nanobody and a diabody.

In another embodiment, the TSLP antagonist is a soluble TSLPR receptor. See, e.g., Al-Shami et al., JEM 202:829-839 (2005).

In another embodiment, the TSLP antagonist is a TSLPR-Fc fusion protein. See, e.g., Zhang et al., Clin. Exp. Immunol. 164:256-264 (2011).

### IV. Determination of Expression Levels of Biomarkers

The methods described herein are generally applicable to determining the expression levels of biomarkers and can be used to measure the expression level of IL-19.

In one aspect, the invention involves determining whether a sample from a subject exhibits increased or decreased levels of a biomarker compared with control levels. Biomarker levels can be quantitated by any method known in the art, including but not limited to, mass spectrometry, Western blot, IHC or ELISA. Means for determining the level of the biomarker of the present invention include, but are not limited to, the methods disclosed herein, and their equivalents. Determination of the level of biomarkers at the protein level, as opposed to at the gene expression level, is convenient because protein can be detected non-invasively or by minimally invasive procedures, such as in plasma and feces.

In one embodiment, biomarker protein levels are determined by Western blot (immunoblot), for example as follows. A biological sample is electrophoresed on 10% sodium dodecyl sulfate polyacrylamide gel (SDS-PAGE) and transferred (e.g. electroblotted) onto nitrocellulose or polyvinylidene fluoride (PVDF) some other suitable membrane. The membrane is then incubated with a primary antibody that binds to the biomarker protein being evaluated, washed, and optionally incubated with a detectably labeled secondary antibody that binds to the primary antibody, and optionally washed again. The presence of the secondary antibody is then detected (or primary antibody if it is detectably labeled), for example by radioactivity, fluorescence, luminescence, enzymatic activity (e.g. alkaline phosphatase or horseradish peroxidase) or other detection or visualization technique known to those of skill in the art. In one embodiment, the detectable label is used to produce an autoradiograph, which is scanned and analyzed. In other embodiments, the gel is imaged directly without the use of an autoradiograph. Observed biomarker band intensity may optionally be normalized to a control protein present in the sample, such as actin or tubulin.

In yet another embodiment, biomarker levels are determined by ELISA. In one embodiment, the sandwich ELISA, a first antibody specific for the biomarker of interest (the “capture antibody”) is coated in the well of a plate (e.g. a 96-well microtiter plate), and the plate is then blocked with, e.g., bovine serum albumin (BSA) or casein. Standards or samples are pipetted into the wells so that biomarker polypeptide present in the samples can bind to the immobilized antibody. The wells are washed and a (second) biotinylated anti-biomarker antibody is added. This second anti-biomarker antibody must be able to bind to the biomarker even while the biomarker is bound to the first antibody. In other embodiments, the second antibody is the same as the first antibody, for example if the biomarker forms a multimer. In some embodiments the second antibody is a distinct, non-crossreacting antibody. In yet other embodiments the second antibody binds to an entirely separate polypeptide chain, for example when the biomarker to be detected is present as a heterodimeric complex (e.g. calprotectin). After washing away unbound biotinylated antibody, HRP-conjugated streptavidin (or some functionally equivalent detection reagent) is pipetted to the wells. Alternatively, the biotinylated antibody can be replaced with an antibody having a directly detectable label, obviating the need for the streptavidivn step. The wells are again washed, a TMB substrate solution is added to the wells, and color develops in proportion to the amount of biomarker bound. Stop solution is added to the reaction, which changes the color from blue to yellow, and the intensity of the color is measured at 450 nm. See e.g., Human IGF-BP-2 ELISA Kit from RayBiotech, Inc.; Norcross, Ga., USA; and Angervo et al., (1992) Biochem. Biophys. Res. Comm. 189: 1177; Kratz et al. (1992) Exp. Cell Res. 202: 381; and Frost et al. (1991) J. Biol. Chem. 266: 18082. A standard curve using known concentrations of biomarker can be used to determine the the concentration of biomarker in the sample.

Other ELISA formats familiar to those in the art may also be used, such as using direct adsorption to the plate, rather than a capture antibody, to immobilize the biomarker in the microtiter well. Competitive ELISA may also be used, in which a biomarker in a sample is detected by its ability to compete with labeled biomarker molecules present in the assay solution for binding to the plate. The higher the concentration of biomarker polypeptide in the sample of interest, the more it will block the binding of labeled biomarkers, thus lowering the observed signal.

Lateral flow format immunoassays (immunochromatographic assay) may also be used, in which an aqueous sample is drawn over a surface by capillary action. The surface has a first zone in which is deposited a detection reagent (such as a detectably labeled antibody) and a second zone comprising an immobilized capture reagent (e.g. an antibody). Both the capture reagent and detection reagent specifically bind to the biomarker of interest. As the sample flows across the first zone the detection reagent is solubilized and binds to any analyte (biomarker) present in the sample to form a complex. As the sample continues to flow it contacts the second zone, where any complexes are bound to the capture reagent and concentrated. When a colored particle is used as the detectable label, the concentration of particles at the second zone results in a visible color signal. The level of analyte (biomarker) may then be assessed qualitatively or quantitatively by the intensity of the signal at the second zone.

Biomarker levels may also be determined by Radioimmunoassay (RIA). RIA involves mixing known quantities of radioactive analyte (e.g., labeled with ¹³¹I and ¹²⁵I-tyrosine) with antibody to that analyte, in the presence or absence of unlabeled or “cold” analyte from a sample of interest, and measuring the amount of labeled analyte displaced. In this case the analyte is a biomarker of the present invention. Analyte in the sample will compete with labeled analyte and reduce its binding to the antibody. Unbound analyte is removed, and labeled bound analyte is quantitated. Unbound analyte can be removed, for example, by precipitating the analyte-antibody complexes with a secondary antibody directed against the primary antibody. In another embodiment, the analyte-specific antibodies can be immobilized on the walls of a test tube or microtiter well or to some other solid substrate, so that unbound analyte can be simply washed away.

Any other suitable assay format may be used to detect the biomarker of interest, such as nephelometry/turbidimetry, specifically immunoturbidimetry, which involves measurement of light scattering caused by suspended insoluble antigen (biomarker)/antibody complexes. See, e.g. U.S. Pat. No. 4,605,305. Other methods include radial immunodiffusion (RID), which is observation of a precipitin ring generated by complex formation between an antigen (biomarker) and an antibody, e.g. in an agar/agarose slab. See, e.g. U.S. Pat No. 3,947,250. Such formats are commonly used in clinical assays.

In other embodiments, the iomarker may be detected by mass spectrometric methods. Mass spectrometric methods include time-of-flight, magnetic sector, quadrupole filter, ion trap, ion cyclotron resonance, electrostatic sector analyzer and hybrids of these. In such embodiments, the biomarker in the sample can be identified and quantified using isotope labeled identical synthetic peptides spiked into the sample. In one embodiment, the mass spectrometer is a laser desorption/ionization mass spectrometer. In laser desorption/ionization mass spectrometry, analytes are placed on the surface of a mass spectrometry probe, which presents an analyte for ionization. A laser desorption mass spectrometer employs laser energy, typically from an ultraviolet or infrared laser, to volatilize and ionize analytes for detection by the ion optic assembly. In another mass spectrometric embodiment, the sample is optionally chromatographically fractionated, and biomarker is then captured on a bio-affinity resin, e.g. a resin derivatized with an antibody. The biomarker is then eluted from the resin and analyzed by MALDI, electrospray, or another ionization method for mass spectrometry. In yet another embodiment, the sample is fractionated on an anion exchange resin and detected directly by MALDI or electrospray mass spectrometry.

In other embodiments, the level of gene expression of biomarker genes may be determined. Gene expression at the nucleic acid level can be quantitated by any method known in the art, including but not limited to, Northern blot analysis, gene chip expression analysis, or RT-PCR (real-time polymerase chain reaction). See e.g., Smith et al. (1993). J. Clin. Endocrin. Metab. 77(5): 1294; Cohen et al. (1993) J. Clin. Endocrin. Metab. 76(4): 1031; Dawczynski et al. (2006) Bone Marrow Transplant. 37:589; and Clemmons et al. (1991) J. Clin. Endocrin. Metab. 73:727.

Northern blot analysis is a standard method for detection and quantitation of mRNA. RNA is isolated from a sample to be assayed (e.g., colonic mucosa). RNA is separated by size by electrophoresis in an agarose gel under denaturing conditions, transferred to a membrane, crosslinked, and hybridized with a labeled probe. In one embodiment of the invention, Northern blot analysis involves radiolabeled or nonisotopically detectably labeled nucleic acids as hybridization probes. In one embodiment of the invention, the membrane holding the RNA sample is prehybridized, or “blocked,” prior to probe hybridization to reduce non-specific background. Unhybridized probe is removed by washing. The stringency of the wash may be adjusted as is well understood in the art. If a radiolabeled (or luminescent) probe is used, the blot can be exposed to film for autoradiography e.g, in the presence of a scintillant. If a nonisotopic probe is used, the blot must typically be treated with nonisotopic detection reagents to develop the detectable probe signal prior to film exposure. The relative levels of expression of the genes being assayed can be quantified using, for example, densitometry or visual estimation. The observed expression level may be normalized to the expression level of an abundantly expressed control gene (e.g. ubiquitin).

In another embodiment, biomarker expression is determined using a gene chip (probe array). A biological sample of interest is prepared and hybridized to the chip, which is subsequently washed, stained and scanned. The data are then processed. Target preparation may entail preparing a biotinylated target RNA from the sample to be tested. The target hybridization step may involve preparing a hybridization cocktail, including the fragmented target, probe array controls, BSA, and herring sperm DNA. In one embodiment, the target is hybridized to the probe array for 16 hours, which probe is washed, stained with streptavidin phycoerythrin conjugate and scanned for light emission at 570 nm. The amount of light emitted at 570 nm is proportional to the target bound at each location on the probe array. Computer analysis using commercially available equipment and software is possible (Affymetrix, Santa Clara, Calif., USA).

In a different embodiment, biomarker expression is determined using real time PCR (RT-PCR). Design of the primers and probes required for RT-PCR of the biomarkers of the present invention is within the skill in the art, in light of the sequences provided herein. In one embodiment, RNA is isolated under RNAse free conditions and converted to DNA using reverse transcriptase, as is well known in the art. RT-PCR probes depend on the 5′-3′ nuclease activity of (e.g., Taq) DNA polymerase to hydrolyze an oligonucleotide hybridized to the target amplicon (biomarker gene). RT-PCR probe oligonucleotides have a fluorescent reporter dye attached to the 5′ end and a quencher moiety coupled to the 3′ end (or vice versa). These probes are designed to hybridize to an internal region of a PCR product.

During amplification, the 5′-3′ nuclease activity of the polymerase cleaves the probe, decoupling the fluorescent dye from the quencher moiety. Fluorescence increases in each cycle as more and more probe is cleaved. The resulting fluorescence signal is monitored in real time during the amplification on standard, commercially available equipment. The quantity of biomarker RNA in a sample being evaluated may be determined by comparison with standards containing known quantities of amplifiable RNA.

Biomarkers or biomarker gene expression may be detected using commercially available kits, or using custom assays with commercially available anti-biomarker antibodies obtained from suppliers well known in the art, or using custom assays and antibodies raised by the investigator.

One of skill in the art would recognize that the detection means disclosed herein inherently involve the transformation of an article from one state into another state. Typically the detection means disclosed herein involve transforming an analyte (i.e. the substance to be detected, such as a biomarker polypeptide or an mRNA encoding that polypeptide) into a complex with a detection reagent (e.g. an antibody or complementary nucleic acid). For example, immunological detection means like ELISA, Western blot, etc. involve transformation of biomarker polypeptides into antigen-antibody complexes, which complex formation is essential to the detection. In another example, hybridization-based detection means like amplification (e.g. TaqMan®), Southern/Northern blotting and gene chip-based methods involve transformation of an mRNA encoding the biomarker from a single stranded state to a double stranded state, which complex formation is essential to the detection.

In some embodiments of the present invention the samples to be compared will be obtained from the same subject, and thus will be to some degree “internally controlled.” In such embodiments, the ability to discern changes in protein or gene expression levels will be limited only by the inherent precision of the assay, and will not include individual-to-individual variation. Accordingly, small differences between samples from a single subject may be statistically significant even when similar data that include individual-to-individual variation would not be.

### V. Data Analysis

Expression levels of the biomarker of the present invention (IL-19) may be used, depending on the samples being compared, for various purposes, including but not limited to, diagnosing disease, staging patients, monitoring disease status, selecting patients for treatment with an TSLP antagonist, confirming target engagement, and monitoring therapeutic efficacy. Typically, such methods involve comparing the level of IL-19 in sample obtained from a subject of interest (the “subject”) to the level in a “control”. For the biomarkers of the present invention, higher levels correlate with disease, or more severe disease status. As used herein, “level of biomarkers in a subject” and similar phrases refer to levels determined in samples obtained from the subject, e.g. skin, tissue, serum, blood, urine, feces, etc.

In light of the identification of the biomarkers provided herein, it would be within the skill in the art for medical practitioners to determine the levels of the IL-19 in a number of human subjects, both with and without allergic disease. Such data would likely be accumulated in the course of clinical trials assessing the safety and efficacy of a drug (e.g. a TSLP antagonist antibody) in question. Such biomarker data are often collected in the course of clinical trials, and represent no more than the usual level of effort expended in the art. These baseline data would also be analyzed for variability using standard statistical approaches to determine the precision of the assay(s) in question. Armed with the difference in biomarker level, and the statistical variability in the assay used to measure the biomarker, a skilled medical practitioner would be able to judge whether the level of the biomarker in a given sample was consistent with TSLP blockade.

The broad scope of this invention is best understood with reference to the following examples, which are not intended to limit the inventions to the specific embodiments.

### EXAMPLES

### Example 1

### General Methods

Standard methods in molecular biology are described. Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols.1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described. Coligan et al. (2000) Current Protocols in Protein Science, Vol. 1, John Wiley and Sons, Inc., New York. Chemical analysis, chemical modification, post-translational modification, production of fusion proteins, glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available. See, e.g., Coligan et al. (2001) Current Protcols in Immunology, Vol. 4, John Wiley, Inc., New York.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalog, Molecular Probes, Inc., Eugene, Oreg.; Sigma-Aldrich (2003) Catalog, St. Louis, Mo.

Standard methods of histology of the immune system are described. See, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.

Statistical analysis may be performed using commercially available software, including but not limited to JMP® Statistical Discovery Software, SAS Institute Inc., Cary, N.C., USA.

Biomarker levels (for example, the level of IL-19) may be determined using commercially available kits or commercially available antibodies, as detailed in the Examples below. Unless otherwise indicated, commercial kits (such as ELISA kits) are used substantially as suggested by the manufacturer. Alternatively, ELISAs and other immunological assays may be developed using commercially available antibodies, or antibodies may be raised to biomarkers for which suitable antibodies are not commercially available. Biomarker gene expression may be monitored using commercially available probes or primers, or such probes or primers may be custom synthesized based on the nucleic acid sequences disclosed herein (either in the sequence listing or disclosed by accession number and incorporated by reference).

### Example 2

### Calcipotriol-Induced Skin Inflammation is Reduced in IL-19 Knock-Out Mice Treated with an Anti-TSLP Antibody

It has been previously shown that TSLP is induced in keratinocytes after application of calcipotriol, a Vit D3 analogue, to the skin resulting in a disease corresponding to atopic dermatitis (AD). See Li M. et al., Proc. Natl. Acad. Sci. 103:11736 (2006); Li M. et al., J. Invest. Dermatol. 129:498 (2009).

In order to determine whether IL-19 plays a role in the progression of AD, calcipotriol-induced sldn inflammation was compared in wild type mice (C57BL/6) and IL-19 knock out (IL19KO) mice. Groups of mice were given a single dose of neutralizing TSLP antibodies or isotype control (40 mpk of 22E5 (rat anti-mouse TSLP) or 5G5 (rat IgG2a isotype control antibody)) one day before onset of calcipotriol treatment. Calcipotriol was applied daily and ear swelling was assessed as a measurement of inflammation. The anti-TSLP antibody used in this study (22E5) is a rat anti-mouse TSLP antibody (IgG2a/kappa) developed in-house using standard procedures.

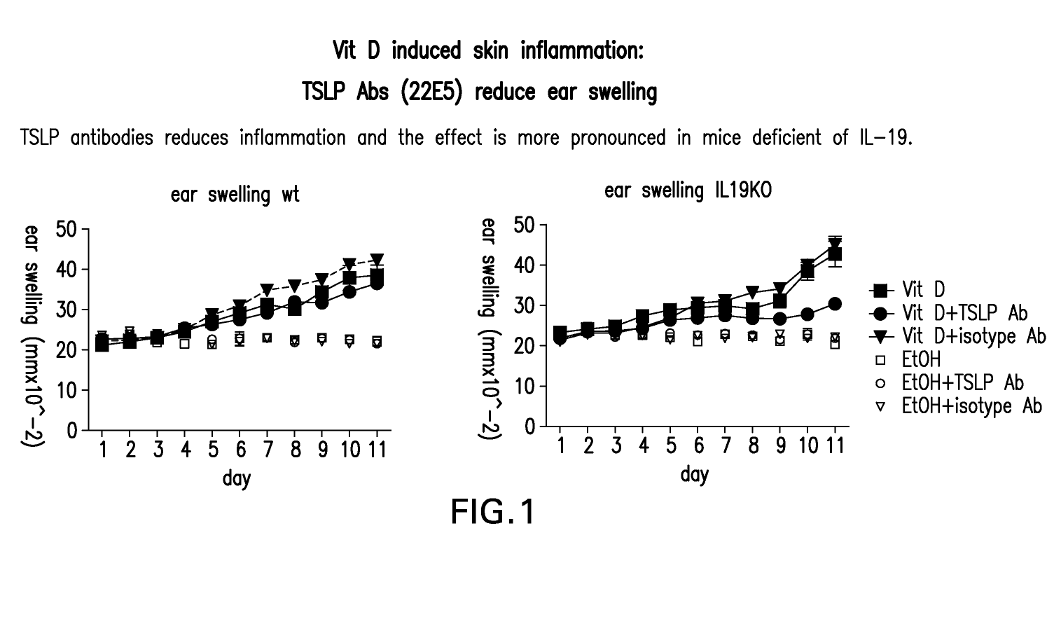

As shown in FIG. 1, IL19KO mice showed reduced ear swelling compared to wt mice. Animals treated with TSLP mAbs also showed a reduction in swelling compared to wt mice, but the greatest reduction in ear swelling was seen in IL19KO mice that also received TSLP antibodies (FIG. 1, right panel).

### Materials and Methods

### Mice

C57BL/6 and Balb/c mice (6-8 weeks) were purchased from Jackson Labs (Bar Harbour, Me.). IL-19 KO mice were generated as described previously (Chan J. R. et al., J. Exp. Med 203:2577 (2003)) and further back-crossed for 10 generations onto C57BL/6 background. All experiments were performed according to institutional guidelines and under IACUC-approved protocols.

### Vitamin D Induced Skin Inflammation

The vitamin D analogue Calcipotriol (Tocris, Ellisville, Mo.) was dissolved in ethanol and was applied daily to both sides of the ears at a fmal concentration of 2 nmol/ear. Control animals were given ethanol only. Ear swelling was measured daily using an engineer's pressure gauge (Peacock, Japan) and blood was collected on days 4 and 8. The blood was spun at 10.000 rpm for 5 minutes to separate the serum, which was frozen at −80 C until analyzed by ELISA. Calcipotriol applications were stopped on day 8 (Balb/c mice) or day 11 (C57BL/6 mice), ears were harvested and fixed in 10% formalin for pathological evaluation or frozen directly in liquid nitrogen for mRNA extraction.

### Example 3

### IL-19 Serum Levels are Reduced in Absence of TSLP

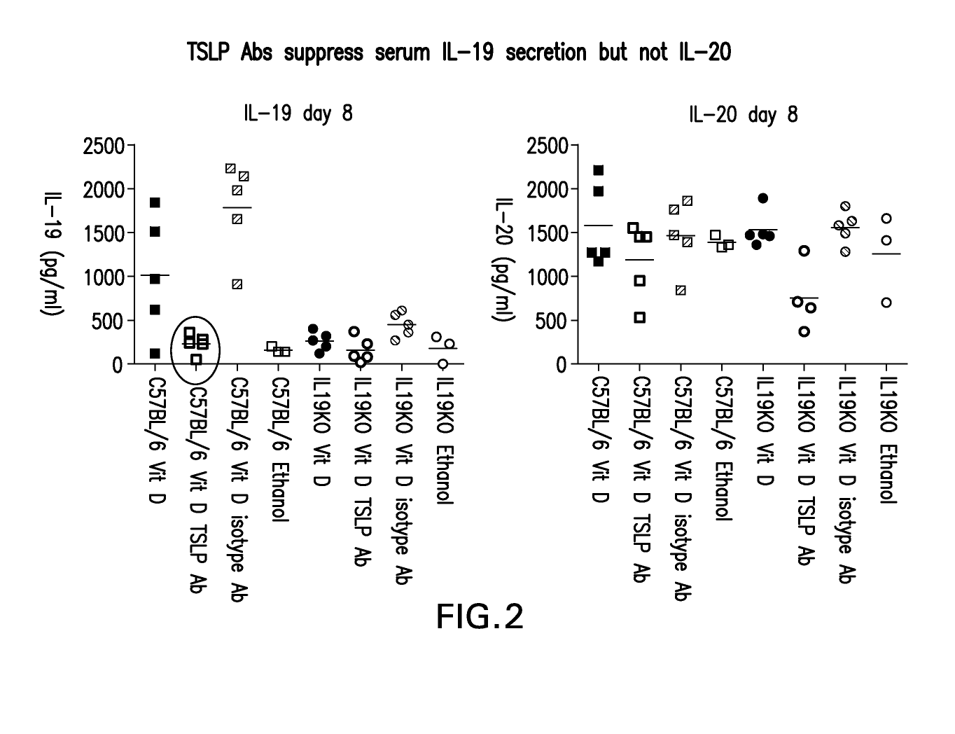

Serum from the animals described in Example 2 was collected on days 4 and 8 of calcipotriol treatment, and assessed by specific ELISA. IL-19 was detected at high levels in the serum of calcipotriol treated mice. Interestingly, mice treated with TSLP antibodies showed a significant reduction in IL-19 serum levels compared with control groups (FIG. 2). Furthermore IL-20, a cytokine of the same family as IL-19 and binding to the same receptor complex as IL-19, did not show the same reduction. In fact, serum levels of IL-20 remained the same between the different treatment groups suggesting that IL-20 is regulated independently of TSLP.

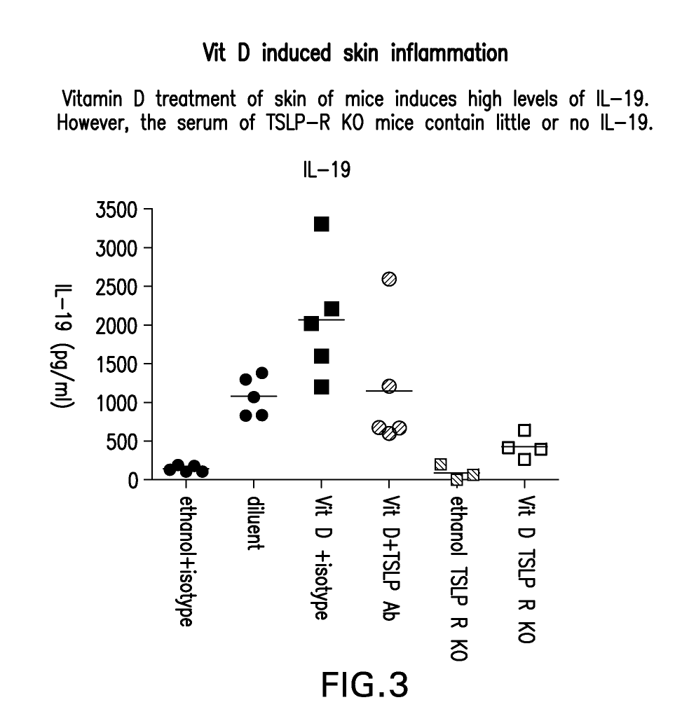

IL-19 was also measured in the serum from TSLP-R KO mice. TSLP-R KO mice (Balb/c background) were obtained from Dr. W. Leonard, NIH, Bethesda, Md. (Al-Shami A. et. al., J. Exp. Med. 200:159-168 (2004)). FIG. 3 shows that TSLPR KO mice have even lower serum levels of IL-19 than calcipotriol treated mice treated with 40 mpk of neutralizing TSLP antibodies (IC8, a rat IgG1 anti-mouse TSLP neutralizing antibody generated in house using standard procedures). Mice treated with calcipotriol but given an isotype control (a rat IgG1 control antibody generated in house and designated 25D2) show no reduction in the levels of IL-19. Control groups treated with ethanol and isotype control (25D2) did not display any inflammation and therefore low levels of IL-19. The lane labeled as “diluent” shows a control consists of 0.1 mg/ml low peroxide Tween 20 in antibody formulation buffer (which is 20 mM NaAcetate, 7% sucrose, pH5.5).

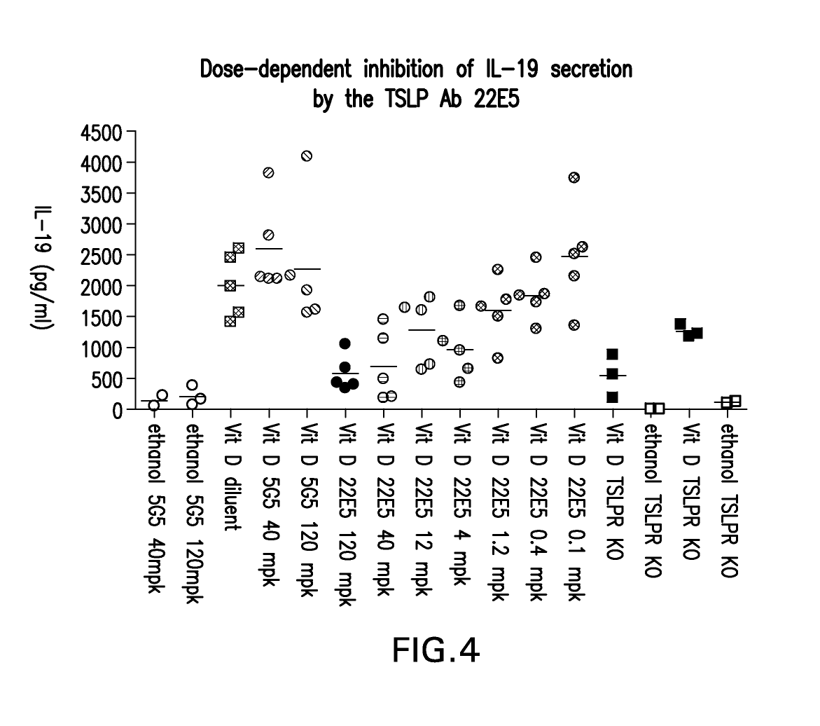

Groups of mice were given a single injection of neutralizing TSLP antibodies (22E5) at various doses (0.1 mpk-120 mpk) one day prior to calcipotriol treatment and serum levels of IL-19 were assessed. FIG. 4 show a clear inhibition of IL-19 at increasing concentrations of blocking TSLP Ab, whereas serum from mice treated with isotype control mAb (5G5) had IL-19 levels similar to calcipotriol treated control mice. FIG. 4 shows that the inhibition by blocking TSLP Ab of IL-19 in serum of calcipotriol treated mice is dose dependent

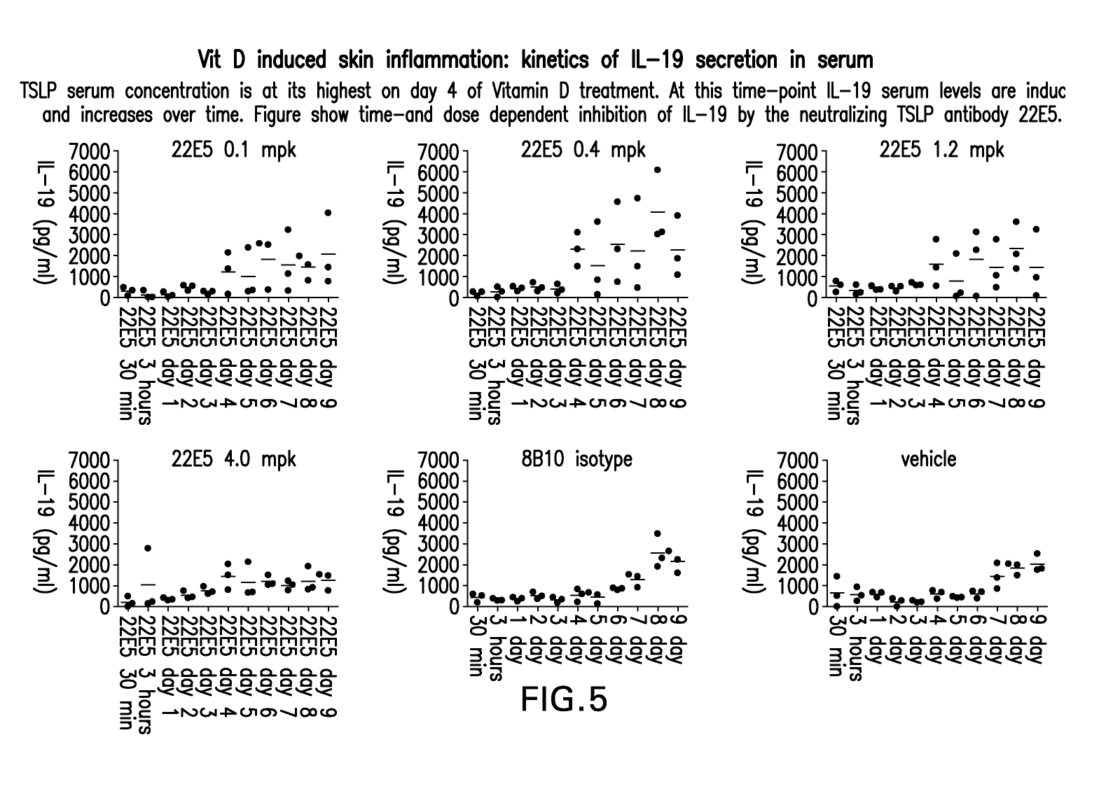

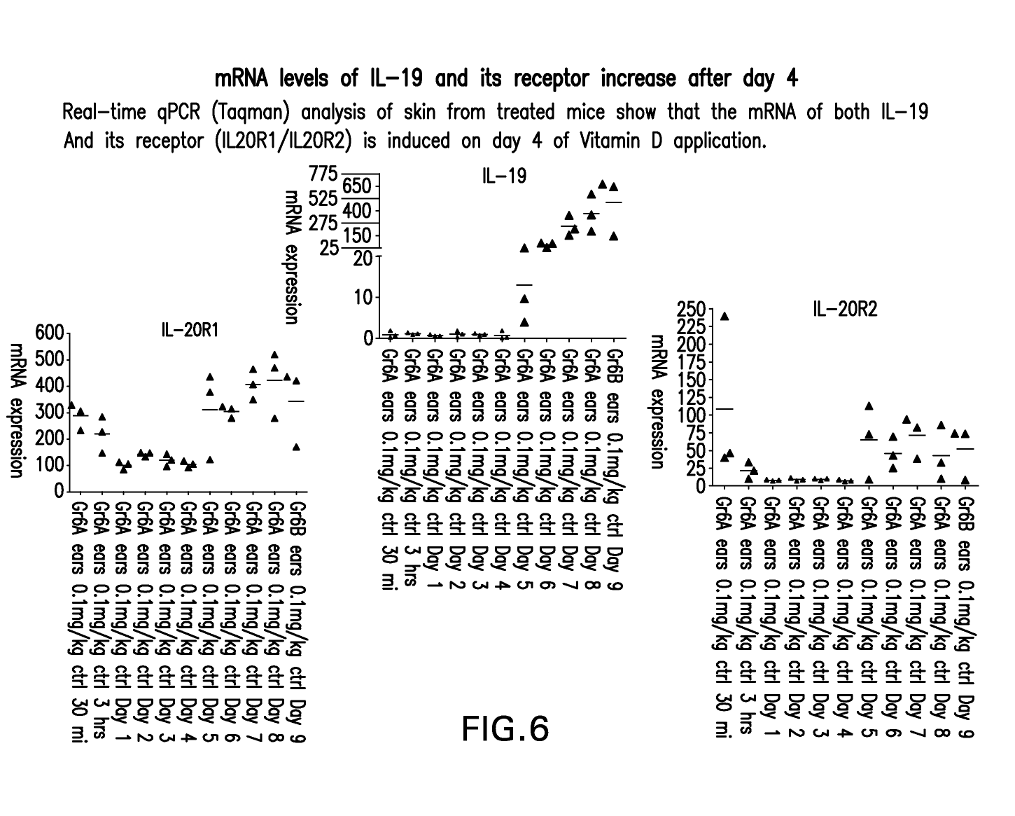

Serum levels of TSLP peak on day 4 of calcipotriol treatment and then slowly decline. To further determine the inter-relationship between IL-19 and TSLP, a dose-and kinetic study was performed using increasing doses of TSLP Abs (0.1 mpk-4 mpk of 22E5) and collecting serum after different time-points from 30 min, 3 hours and then daily from day 1 through day 9. As shown in FIG. 5, in the absence of TSLP, little IL-19 is secreted into the serum. However, when TSLP concentration reaches its peak on day 4, IL-19 secretion is induced and then further increases until day 9. Likewise, mRNA levels in ear skin of treated mice show the same trend and IL-19, as well as its receptor IL-20R1/IL-20R2 are induced on day 4 after calcipotriol treatment, FIG. 6.

### Materials and Methods

### ELISA

ELISA for mouse IL-19 was performed by coating plates (Nunc, Maxi-Sorp Rochester, NY) with 1 ug/ml rat anti-mouse IL-19 mAb (1155, IgG2a, generated in-house) in 100 ul PBS over night at room temperature. After blocking with 1% BSA for 1 hour, plates were washed and murine rIL-19 (R&D Systems, Minneapolis, Minn.) was used as standard. Serum samples were diluted in PBS 0.05% Tween-20 and the plate further incubated on a shaker for 2 hours at room temp. After washing, a polyclonal goat anti-mouse IL-19 biotin (eBioScience, San Diego, Calif.) was added at 1 ug/ml in PBS-Tween. The plate was further incubated for 2 hours, washed, and extravidin-peroxidase (Sigma, St. Louis, Mo.) was added at a 500× dilution. After 30 min. incubation, plates were washed and TMB substrate (Kirkegaard&Perry, Gaitherburg, MD) was added. The reaction was stopped using 1M phosphoric acid, absorbances were read at 450-570nm using a Vmax plate reader (Molecular Devices, Sunnyvale, Calif.) and data were analyzed using SoftMax Pro software. ELISAs detecting mouse IL-20, mouse TSLP and human IL-19 were assessed using kits from R&D Systems. ELISAs were performed according to manufacturers' instructions.

mRNA Extraction and Real-Time Quantitative PCR

RNA extraction was performed using standard techniques, and gene expression was calculated using the Δ-ΔCt method (mean cycle threshold value for ubiquitin—Ct gene of interest)×10̂4 was used to normalize the values.

### Example 4

### Skin Lesions in Cynomolgus Monkey Skin Contains IL-19

To determine whether IL-19 could be detected in species other than mice, skin from cynomolgus monkeys were examined. These monkeys were part of a study on house-dust mite induced asthma (Ayanoglu G. et al., Eur. Respir. J. 37:541 (2011)), and found to develop spontaneous atopic dermatitis skin lesions during the later course of the study. Out of the genes tested, 36 genes—including IL-19—were upregulated in at least one lesion (data not shown). mRNA from cynomolgus monkey lesional skin was then compared to mRNA isolated from calcipotriol treated mouse ears. A few genes were found to be induced in both species, including IL-19.

### Materials and Methods

### Cynomolgus Monkey Skin

Skin samples were obtained from cynomolgus macaques participating in a study on house dust mite (HDM)-induced asthma (Ayanoglu G. et al., Eur. Respir. 37:541 (2011)). With time (>2 years) the animals developed spontaneous lesions resembling atopic dermatitis. Lesional biopsies were taken and snap frozen in liquid nitrogen. Samples were kept at −80 C until processed and analyzed for mRNA expression by Taqman.

### Example 5

### IL-19 in Human Histoculture

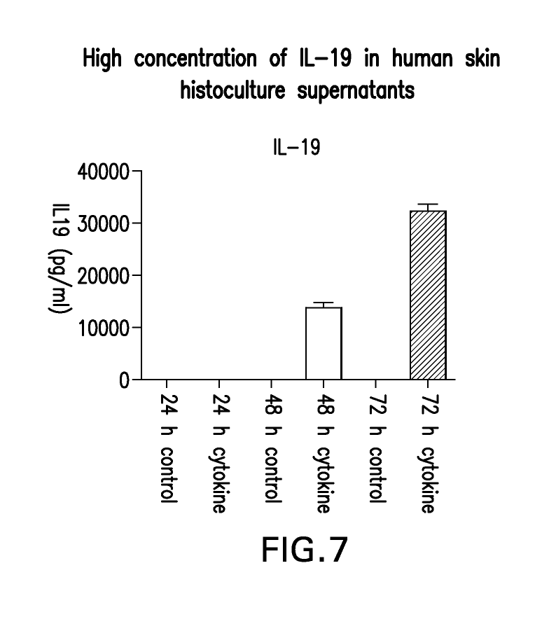

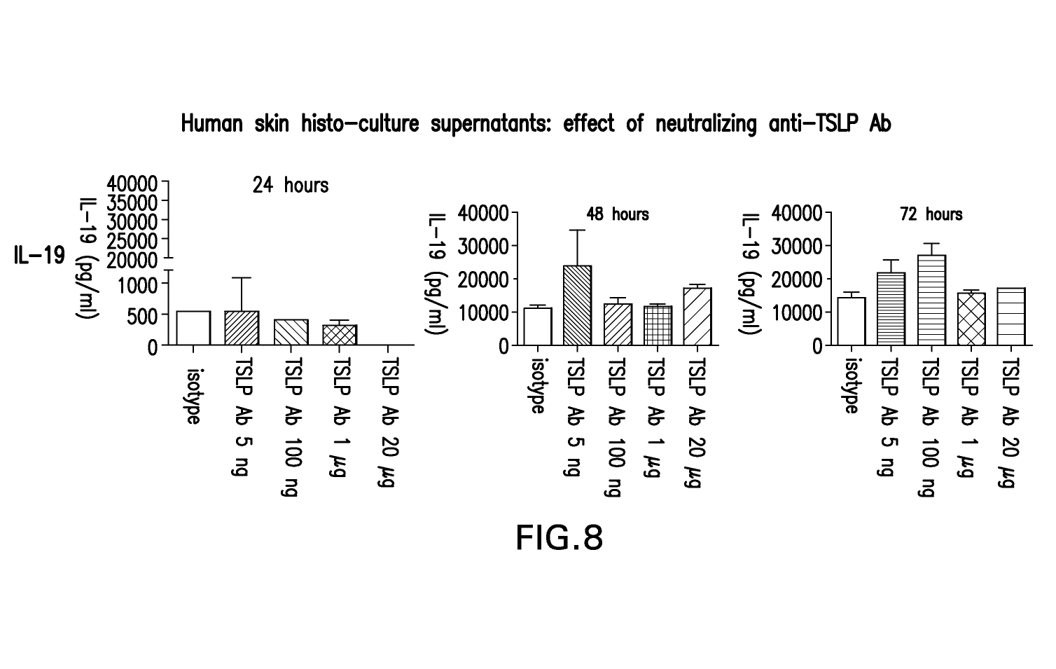

To assess IL-19 in human samples, skin from patients undergoing plastic surgery was cut into pieces of 2-3 mm3 and placed on Millicell tissue culture inserts (histoculture) either in medium alone, or with a cytokine mixture (IL-4, IL-13, TNF-α) that had been shown to promote optimal secretion of TSLP. Supernatants were harvested at different timepoints and analyzed using specific ELISA. As shown in FIG. 7, IL-19 could be readily detected in these supernatants, with a nearly three-fold increase in IL-19 concentration between 48 and 72 hours of culture. Data reveals a time-dependent induction of IL-19 in these cultures.

In a next step, increasing concentrations of neutralizing TSLP antibodies (5 ng/ml-20 ug/ml) were added to cultures containing the cytokine mix, and supernatants were collected at different time points. FIG. 8 shows, that TSLP antibodies at higher concentrations (>1 ug/ml) could significantly reduced the levels of IL-19 in these skin cultures.

### Materials and Methods

### Human Skin and Histoculture

Human skin was obtained from consented donors undergoing plastic surgery (abdominoplasty) at Stanford Hospital. Skin samples were cut into pieces of 2-3 mm3 using a scalpel blade and placed on a 0.4 um Millicell culture insert (Millipore, Billerica, Mass.). The skin tissue pieces were cultured in RPMI 1640 supplemented with antibiotics, 2 mM L-glutamine and 10% fetal calf serum either in medium alone, or supplemented with IL-4 (20 ng/ml), Il-13 (100 ng/ml) and TNF-α (100 ng/ml). Tissues were kept in the air-liquid interphase. Supernatants were harvested at the indicated time-points and kept frozen until analyzed using specific ELISA. In some studies, either neutralizing TSLP antibodies (clone 23B12, humanized IgG1, lot 87ACV generated in house) or isotype control antibodies (Xolair, humanized IgG1, lot 76ABW, Genentech, South San Fransisco, Calif.) were added together with the cytokines.

### Example 6

### Expression of IL-19 and TSLP in Patients Subject to Atopy Patch Tests

Atopic dermatitis patients were enrolled in a protocol in which lesional and non-lesional skin biopsies were obtained, and atopic patch tests (APT) with biopsy read-outs were performed. Each biopsy was divided in two parts: one embedded and used for immunohistochemistry and the other was frozen and used for gene expression analyses.

Because atopic dermatitis patients have IgE bearing Langerhans cells in the epidermis, application of allergen on non-lesional skin (APT) will typically elicit a macroscopically visible eczematous reaction within 48-72 hours at the site of allergen application. This reaction is microscopically characterized by an influx of inflammatory cells, mainly T cells of the Th2 type. The eczematous reaction vanishes automatically after 72-96 hours.

Table 1 shows the IL-19 mRNA expression data from 10 patients enrolled in the study as determined by qPCT. In patients 1, 2, 3, 4, 9 and 10, IL-19 mRNA expression was significantly upregulated in biopsies from lesional skin and from skin exposed to the APT tests. Furthermore, TSLP was detectable by immunohistochemistry in the same biopsies (data not shown). Patients 5, 6, 7 and 8, appear to be non-responders to the APT test and their biopsies taken at 24 hr do not show elevated levels of IL-19. Interestingly, in the same biopsies of these patients (5, 6, 7 and 8), expression of TSLP could not be detected by immunohistochemistry in these biopsy samples.

TABLE 1

| Non-lesional | | Lesional | 24 hr | 48 hr |
|---|---|---|---|---|
| Pt1 | −2.400 | −2.63 | 2.16 | 2.85 |
| Pt2 | −2.200 | −0.64 | 1.75 | 3.07 |
| Pt3 | −2.390 | 1.80 | 2.98 | 3.00 |
| Pt4 | −2.280 | 2.67 | 2.30 | 3.55 |
| Pt5 | −2.350 | 0.62 | −2.24 | 2.21 |
| Pt6 | −2.320 | −2.47 | −2.35 | 1.95 |
| Pt7 | −2.300 | −2.48 | −2.21 | 0.71 |
| Pt8 | −2.340 | 2.63 | 2.28 | −2.44 |
| Pt9 | 0.460 | 0.97 | 3.49 | 2.72 |
| Pt10 | −2.570 | 2.43 | 2.86 | 1.62 |

1. A method of treating allergic disease in a subject in need thereof comprising measuring expression of IL-19 in a sample from said subject and, if IL-19 levels in the sample are higher than a control, administering a therapeutically effective amount of a TSLP antagonist to the subject.

2. A method for treating a subject with a TSLP antagonist comprising:
a) obtaining a first biological sample from the subject prior to administering a dose of a TSLP antagonist;
b) measuring the expression of IL-19 in the baseline biological sample,
c) administering the TSLP antagonist to the subject;
d) obtaining from the subject a second biological sample;
e) measuring the expression of IL-19 in the second sample;
f) comparing the expression of IL-19 in the second biological sample with the expression of IL-19 in the first biological sample,
and, if IL-19 levels are reduced in the second biological sample as compared to the first biological sample, administering a therapeutically effective amount of a TSLP antagonist to the subject.

3. A method for selecting a subject from treatment with a TSLP antagonist comprising measuring expression of IL-19 in a sample from said subject and, if IL-19 levels in the sample are higher than the levels of a control, then administering a therapeutically effective amount of a TSLP antagonist to the subject.

4. (canceled)

5. The method of claim 3, wherein the TSLP antagonist is an anti-TSLP antibody.

6. The method of claim 3, wherein the subject is a human subject suffering from an allergic disease.

7. The method of claim 6, wherein the allergic disease is atopic dermatitis.

8. The method of claim 6, wherein the allergic disease is asthma.

9. The method of claim 3, wherein the sample is tissue sample.

10. The method of claim 3, wherein the sample is a skin biopsy.

11. The method of claim 3, wherein the sample is a blood sample.

12. The method of claim 3, wherein the sample is serum.

13. The method of claim 3, wherein the sample is sputum.

14. The method of claim 3, wherein expression of IL-19 is determined by gene expression analysis or immunoassay.

15. The method of claim 3, wherein expression of IL-19 is determined by an immunoassay selected from the group consisting of ELISA, RIA, Western blot, luminescent immunoassay, fluorescent immunoassay.

16. The method of claim 3, wherein expression of IL-19 is determined by gene expression analysis, wherein said gene expression analysis is selected from the group consisting of is selected from the group consisting of Northern blotting, PCR-based, SAGE, flow cytometry-based, and DNA microarray.

17. The method of any one of claim 3, wherein the control is one or more samples from samples from subjects that do not suffer from allergic disease.

18. The method of claim 3, wherein the control is one or more samples from samples from subjects that do not suffer from allergic disease and are not treated with a TSLP antagonist.
